(19) United States
(12) Patent Application Publication
(10) Pub. No.: US 2026/0141601 A1
(43) Pub. Date: May 21, 2026

(54) SYSTEMS AND METHODS FOR ANGIOGRAPHY USING MULTI-ENERGY DETECTORS

(71) Applicant: THE JOHNS HOPKINS UNIVERSITY, Baltimore, MD (US)

(72) Inventors: Jianan GANG, Baltimore, MD (US); Joseph Webster STAYMAN, Baltimore, MD (US)

(21) Appl. No.: 19/119,499

(22) PCT Filed: Oct. 17, 2023
(86) PCT No.: PCT/US2023/035270

§ 371 Date: Apr. 9, 2025

Related U.S. Application Data
(60) Provisional application No. 63/380,053, filed on Oct. 18, 2022.

(51) Int. Cl. G06T 12/30; A61B 6/00; A61B 6/03; A61B 6/42; G06T 5/50; G06T 7/00; G06T 7/254

(52) U.S. Cl. CPC G06T 12/30; A61B 6/032; A61B 6/4208; A61B 6/4241; A61B 6/481; A61B 6/5205; A61B 6/5264; G06T 5/50; G06T 7/0016; G06T 7/254; G06T 2207/30101; G06T 2207/30196; G06T 2211/404; G06T 2211/408

(57) ABSTRACT

Techniques for generating a plurality of basis images for use in angiography are presented. The techniques include: acquiring at least four radiological measurements of a target image region, where the radiological measurements include: a pre-injection measurement set including at least a first pre-injection measurement and a second pre-injection measurement and a post-injection measurement set including at least a first post-injection measurement and a second post-injection measurement; decomposing, using a joint estimate, the radiological measurements into at least four basis images, where the basis images include: a pre-injection first material basis image, a post-injection first material basis image, a second material basis image, and a contrast agent basis image, where the decomposing includes performing at least three-material decomposition using all four of the radiological measurements; and providing at least the contrast agent basis image.

### CROSS-REFERENCE TO RELATED APPLICATIONS

This application is the national stage entry of International Patent Application No. PCT/US2023/035270, filed on Oct. 17, 2023, and published as WO 2024/086131 A1 on Apr. 25, 2024, which claims the benefit of U.S. Provisional Patent Application No. 63/380,053, filed 18 Oct. 2022, which are hereby incorporated by reference herein in their entireties.

### GOVERNMENT SUPPORT

This invention was made with government support under contract EB030494 awarded by the National Institutes of Health. The government has certain rights in the invention.

### FIELD

Systems and methods consistent with the present disclosure are directed to angiography.

### BACKGROUND

Digital Subtraction Angiography (DSA) is a well-established modality for improving visualization of contrast-enhanced vascular structures. The most common technique for DSA is temporal subtraction where a pre-contrast image (or “mask”) is subtracted from subsequent images acquired post-contrast injection. One of the main challenges to this technique is misregistration artifacts resulting from patient motion. Although gross patient motion is less common, involuntary soft tissue motion still degrades the diagnostic values of DSA images. Another technique, energy subtraction, has been proposed to mitigate patient motion. Such method relies on rapid kV switching or energy-resolving detectors to acquire (nearly) simultaneous measurements at different x-ray energies, allowing for material decomposition of soft tissue and bone basis images without relying on a pre-contrast image. While misregistration can be minimized using this method, isolating the contrast agent is challenging. The resulting DSA image is presented with both iodine and bone, or iodine and soft tissue (if using a linear combination method for material decomposition), which reduces diagnostic capabilities due to overlapping anatomy.

A hybrid subtraction method has also been proposed that utilizes spectral measurements acquired both pre- and post injection. See W. R. Brody, Radiology, vol. 141, no. 3, pp. 828-831, 1981. Material decomposition of soft tissue and bone is performed at both time points. The pre-contrast “bone” image contains calcium signals, while the post-contrast bone image contains both calcium and iodine. A subtraction of the “bone” basis image can therefore eliminate bone structures, under the assumption that there is minimal bone movement. Both the energy and hybrid subtraction methods suffer from poor image quality due to the noise amplifying nature of the separate material decompositions. Neither technique has enjoyed widespread adoption since their proposal in the 1980s. In recent years, the advancement in spectral imaging and detector technology has inspired renewed interest in energy subtraction DSA, e.g., using energy resolving detectors for DSA.

### SUMMARY

Various embodiments include methods and systems that use spectral information in the pre-contrast, or previous post-contrast images in a time series, thereby eliminating motion artifact and produce images of improved image quality.

According to various embodiments, method for generating a plurality of basis images for use in angiography is presented. The method includes: acquiring at least four radiological measurements of a target image region, where the at least four radiological measurements include: a pre-injection measurement set including at least a first pre-injection measurement and a second pre-injection measurement; and a post-injection measurement set including at least a first post-injection measurement and a second post-injection measurement; decomposing, using a joint estimate, the at least four radiological measurements into at least four basis images, the at least four basis images including: a pre-injection first material basis image, a post-injection first material basis image, a second material basis image, and a contrast agent basis image, where the decomposing the at least four radiological measurements into at least four basis images includes performing at least three-material decomposition using all of the at least four radiological measurements; and providing at least the contrast agent basis image.

Various optional features of the above method embodiments include the following. The first material may include water, and the second material may include calcium. The contrast agent may include iodine. The acquiring may include acquiring using a multi-energy detector, where the multi-energy detector includes a dual-layer flat panel detector, where the dual-layer flat panel detector includes at least a top layer detector and a bottom layer detector, where the first pre-injection measurement includes a pre-injection bottom layer detector measurement and where the second pre-injection measurement includes a pre-injection top layer detector measurement, and where the first post-injection measurement includes a post-injection bottom layer detector measurement and where the second post-injection measurement includes a post-injection top layer detector measurement. The performing at least three-material decomposition using all of the at least four radiological measurements may include computing a plurality of line integrals of material density, where the plurality of line integrals of material density include: line integrals of first material density, line integrals of second material density, and line integrals of contrast agent density. The performing at least three-material decomposition using all of the at least four radiological measurements may include: decomposing the pre-injection measurement set into at least the pre-injection first material basis image and the second material basis image using a first lookup table; and decomposing the post-injection measurement set into at least the post-injection first material basis image and the contrast agent basis image using a second lookup table, where the first lookup table maps line integrals of first material density and line integrals of second material density to pre-injection intensity measurements from the top layer detector and the bottom detector layer, and where the second lookup table includes a three-material lookup table that maps the line integrals of first material density and line integrals of contrast agent density to post-injection intensity measurements from the top layer detector and the bottom detector layer, indexed according to the line integrals of second material density. The performing at least three-material decomposition using all of the at least four radiological measurements may include: estimating all of the at least four basis images simultaneously using a penalized likelihood objective function. The performing at least three-material decomposition using all of the at least four radiological measurements may further assume that the at least four radiological measurements of the target image region follow a multivariate Gaussian distribution with a covariance. The acquiring may include acquiring using a triple-layer flat panel detector. The acquiring may include acquiring using a photon counting detector. The at least four radiological measurements may further include an additional measurement obtained prior to the post-injection measurement set, and the decomposing the at least four radiological measurements into at least four basis images may further include performing at least three-material decomposition using all of the at least four radiological measurements and the additional measurement. The method may include providing at least one of the pre-injection first material basis image, the post-injection first material basis image, or second material basis image.

According to various embodiments, a system for generating a plurality of basis images for use in angiography is presented. The system includes: at least one computer; at least one electronic storage device; the at least one electronic storage device storing instructions that, when executed by the at least one computer, cause the at least one computer to generate a plurality of basis images by performing actions including: acquiring at least four radiological measurements of a target image region, where the at least four radiological measurements include: a pre-injection measurement set including at least a first pre-injection measurement and a second pre-injection measurement; and a post-injection measurement set including at least a first post-injection measurement and a second post-injection measurement; decomposing, using a joint estimate, the at least four radiological measurements into at least four basis images, the at least four basis images including: a pre-injection first material basis image, a post-injection first material basis image, a second material basis image, and a contrast agent basis image, where the decomposing the at least four radiological measurements into at least four basis images includes performing at least three-material decomposition using all of the at least four radiological measurements; and providing at least the contrast agent basis image.

Various optional features of the above system embodiments include the following. The first material may include water, and where the second material may include calcium. The contrast agent may include iodine. The system may further include a multi-energy detector, where the acquiring includes acquiring using the multi-energy detector, where the multi-energy detector includes a dual-layer flat panel detector, where the dual-layer flat panel detector includes at least a top layer detector and a bottom layer detector, where the first pre-injection measurement includes a pre-injection bottom layer detector measurement and where the second pre-injection measurement includes a pre-injection top layer detector measurement, and where the first post-injection measurement includes a post-injection bottom layer detector measurement and where the second post-injection measurement includes a post-injection top layer detector measurement. The performing at least three-material decomposition using all of the at least four radiological measurements may include computing a plurality of line integrals of material density, where the plurality of line integrals of material density include: line integrals of first material density, line integrals of second material density, and line integrals of contrast agent density. The performing at least three-material decomposition using all of the at least four radiological measurements may include: decomposing the pre-injection measurement set into at least the pre-injection first material basis image and the second material basis image using a first lookup table; and decomposing the post-injection measurement set into at least the post-injection first material basis image and the contrast agent basis image using a second lookup table, where the first lookup table maps line integrals of first material density and line integrals of second material density to pre-injection intensity measurements from the top layer detector and the bottom detector layer, and where the second lookup table includes a three-material lookup table that maps the line integrals of first material density and line integrals of contrast agent density to post-injection intensity measurements from the top layer detector and the bottom detector layer, indexed according to the line integrals of second material density. The performing at least three-material decomposition using all of the at least four radiological measurements may include: estimating all of the at least four basis images simultaneously using a penalized likelihood objective function. The performing at least three-material decomposition using all of the at least four radiological measurements may further assume that the at least four radiological measurements of the target image region follow a multivariate Gaussian distribution with a covariance. The system may further include a triple-layer flat panel detector, where the acquiring includes acquiring using the triple-layer flat panel detector. The system may further include a photon counting detector, where the acquiring includes acquiring using the photon counting detector. The at least four radiological measurements may further include an additional measurement obtained prior to the post-injection measurement set, and the decomposing the at least four radiological measurements into at least four basis images may further include performing at least three-material decomposition using all of the at least four radiological measurements and the additional measurement. The actions may further include providing at least one of the pre-injection first material basis image, the post-injection first material basis image, or second material basis image.

Additional features and embodiments of the invention will be set forth in part in the description which follows, and in part will be obvious from the description, or may be learned by practice of the invention. It is to be understood that both the foregoing general description and the following detailed description are exemplary and explanatory only and are not restrictive of the claimed subject matter.

### BRIEF DESCRIPTION OF THE DRAWINGS

The accompanying drawings, which are incorporated in and constitute a part of this specification, illustrate exemplary embodiments and together with the description, serve to explain the principles of the disclosure. In the figures:

FIG. 1 is a schematic diagram of a system for generating a plurality of basis images for use in angiography, according to various embodiments;

FIG. 2 depicts a four-material decomposition consistent with this disclosure using a lookup table approach;

FIG. 3 depicts a four-material decomposition consistent with this disclosure using a model-based approach;

FIG. 4 depicts an improvement, according to reductions to practice, relative to a prior art approaches; and

FIG. 5 is a flow diagram for a method of generating a plurality of basis images for use in angiography, according to various embodiments.

### DESCRIPTION OF THE EMBODIMENTS

Reference will now be made in detail to disclosed embodiments, examples of which are illustrated in the accompanying drawings.

Various embodiments include a method and system that allows for three-material decomposition, e.g., using a dual-layer flat panel detector, in the presence of soft tissue motion. According to various embodiments, the calcium signal (bone) may remain stationary in the pre- and post-contrast images, but the soft tissues may move freely (e.g., cardiac motion). The dual-layer pre- and post-injection images may form an ensemble of four radiological measurements that permits material decomposition of four bases (e.g., pre- and post-injection soft tissue, calcium, and iodine).

According to various embodiments, instead of performing material decomposition for the pre- and post measurements separately, four material bases-soft tissue pre-contrast, bone, soft tissue post-contrast, and iodine—may be jointly estimated using all four measurements. Such formulation allows three-material decomposition to be performed, e.g., with a dual-layer detector, and also offers image quality advantages compared to the conventional hybrid subtraction method.

Moreover, joint estimation according to various embodiments utilizes both temporal and spectral information for the material decomposition, reducing artifacts and improving accuracy. For example, according to some embodiments, a pre-injection soft tissue measurement as well as a post-injection soft tissue measurement may both be taken into account in the decomposition. This temporal information, as well as the spectral information (e.g., for water/soft tissue, calcium/bone, and contrast agent), when used for three-material decomposition, provides more accurate results in comparison to approaches where the decomposition is performed solely using spectral information, even if the spectral information is obtained at multiple times (e.g., pre-injection and post-injection).

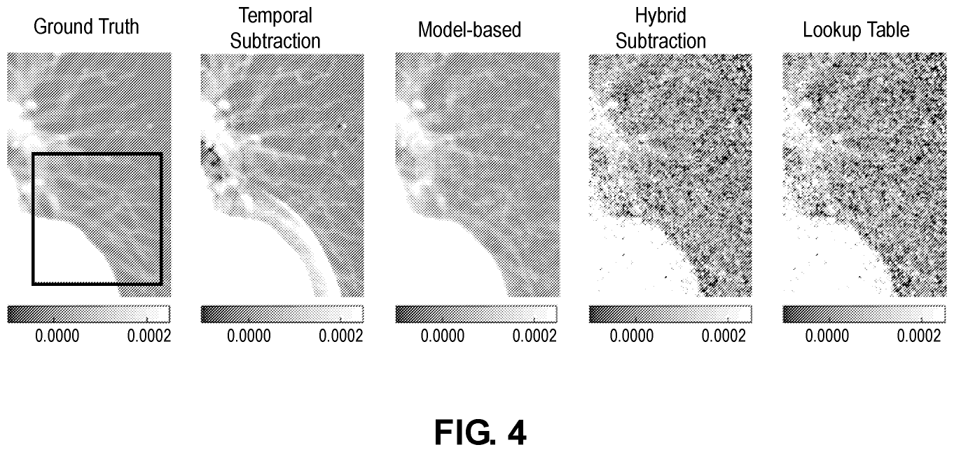

Various embodiments may utilize either of two different processing techniques: (1) a modified lookup table; or (2) a model-based material estimation. Further, this disclosure compares reductions to practice that employed these processing techniques to the results of previously-proposed DSA methods using temporal subtraction and hybrid (dual-energy) subtraction, as shown and described in reference to FIG. 4. Investigations were performed using an XCAT thorax phantom simulating a breath-hold. The pre- and post-contrast measurements were simulated at different time points within a cardiac cycle.

Both the lookup table and model-based approaches can eliminate motion artifacts as a result of soft tissue motion and allow good separation of iodine, bone, and soft tissue. While the lookup table algorithm can contain high noise at the simulated dose level, which can be corrected by adjusting the dose level or utilizing post processing, the model-based algorithm produced iodine images that allowed for the visualization of major vessels around the heart. By contrast, as illustrated in FIG. 4, traditional temporal DSA is susceptible to subtraction artifacts, and hybrid DSA shows increased noise.

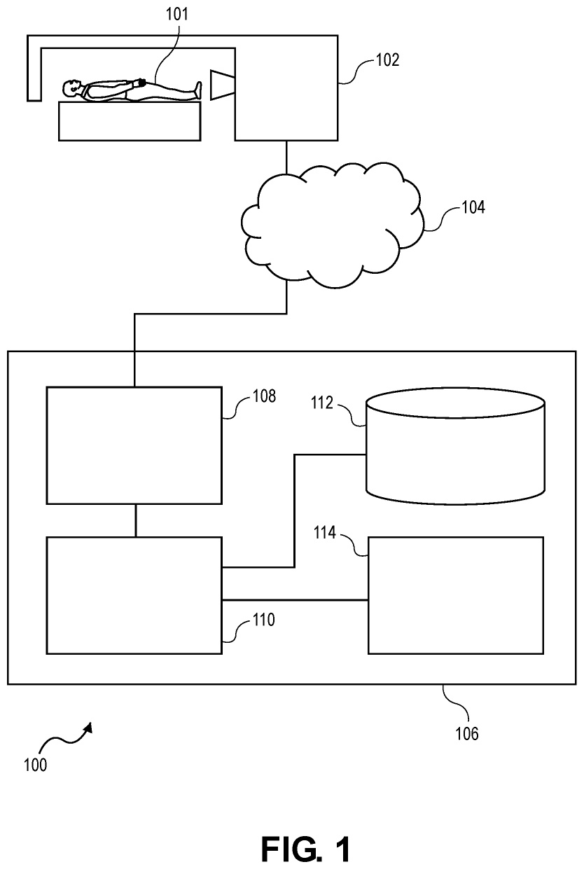

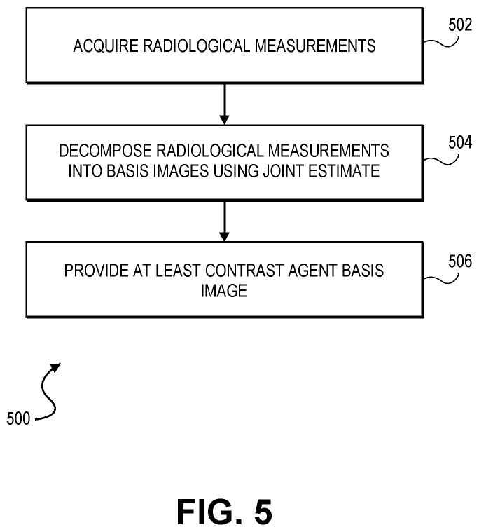

FIG. 1 is a schematic diagram of a system 100 for generating a plurality of basis images for use in angiography, according to various embodiments. The system 100 includes an x-ray machine 102 and a computer system 106. The system 100 may be configured to perform a method of generating a plurality of basis images for use in angiography, e.g., the method 500 as shown and described herein in reference to FIG. 5.

The system 100 includes x-ray machine 102. The x-ray machine 102 is configured to acquire radiological measurements of an anatomical feature of a patient 101. Such radiological measurements may be captured prior to and subsequent to the patient 101 being injected with a contrast agent. Note that although example embodiments are described herein with respect to iodine as a contrast agent, embodiments are not so limited. Any contrast agent may be utilized, not limited to iodine. By way of non-limiting examples, other suitable contrast agents include gold nanoparticles or barium.

Any of a variety of x-ray machines may be used according to various embodiments. According to some embodiments, the x-ray machine 102 may include a multi-energy detector, where the multi-energy detector includes a dual-layer flat panel detector, with the bottom layer detecting higher energy x-rays and the bottom layer detector detecting lower energy x-rays. According to some embodiments, the x-ray machine 102 may include a triple layer detector, where the a triple layer detector includes a three-layer flat panel detector, with the bottom layer detecting higher energy x-rays, the middle layer detecting intermediate energy x-rays, and the bottom layer detector detecting lower energy x-rays. According to some embodiments, the x-ray machine 102 may include two or more photon counting detectors. According to some embodiments, the x-ray machine 102 is configured for rapid kV switching and/or includes energy-resolving detectors to acquire (nearly) simultaneous measurements at different x-ray energies.

The x-ray machine 102 is communicatively coupled to a computer system 106, either directly or via a network 104, as shown. The computer system 106 includes an input interface 108 through which data representing radiological measurements is received. The input interface 108 is communicatively coupled to one or more processors 110.

The processors 110 are communicatively coupled to a random access memory 114 operating under control of or in conjunction with an operating system. The processors 110 in embodiments may be included in one or more servers, clusters, or other computers or hardware resources, or may be implemented using cloud-based resources. The operating system may be, for example, a distribution of the Linux™ operating system, the Unix™ operating system, or other open-source or proprietary operating system or platform. The processors 110 may communicate with data store 112, such as a hard drive or drive array, to access or store program instructions, e.g., to perform methods disclosed herein, and other data. Processors 110 may, in general, be programmed or configured to execute control logic and control operations to implement methods disclosed herein, e.g., the method 500. Other configurations of computer system 100, associated network connections, and other hardware, software, and service resources are possible.

In general, various embodiments may acquire an initial pre-contrast injection radiological measurement, e.g., a projection image, followed by a post-injection radiological measurement, e.g., a projection image. Both exposures may use a dual-layer flat-panel detector, yielding a total of four images. Various embodiments may assume there is minimal motion of bone between the pre-contrast and post-contrast acquisitions, or that bone motion can be compensated via registration. The calcium distribution in the pre- and post-contrast images can therefore be assumed to be unchanged. According to various embodiments, the forward model for the four pre- and post-contrast measurements, y, can be written as follows, by way of non-limiting example:

$$\begin{matrix}{\overline{y}}_{1} = {B}_{t} {S}_{t} exp ( - {Q}^{{H}_{2} O} {l}_{1}^{{H}_{2} O} - {Q}^{Ca} {l}^{Ca} ) & {\overline{y}}_{2} = {B}_{b} {S}_{b} exp ( - {Q}^{{H}_{2} O} {l}_{1}^{{H}_{2} O} - {Q}^{Ca} {l}^{Ca} )\end{matrix} \qquad ( 1 )$$

$${\overline{y}}_{3} = {B}_{t} {S}_{t} exp ( - {Q}^{{H}_{2} O} {l}_{2}^{{H}_{2} O} - {Q}^{Ca} {l}^{Ca} - {Q}^{{I}_{l} I} ) \qquad {\overline{y}}_{4} = {B}_{b} {S}_{b} exp ( - {Q}^{{H}_{2} O} {l}_{2}^{{H}_{2} O} - {Q}^{Ca} {l}^{Ca} - {Q}^{{I}_{l} I} )$$

In Eq. 1, the operator B denotes detector blur, S denotes the spectral response (including effects of both x-ray source spectrum and spectral sensitivity of the detector), and Q denotes the mass attenuation coefficient that scales the line integrals of material density, [symbol] . The superscripts denote the materials. Subscripts t and b denote top and bottom detector layers, respectively. (Although measurement by way of top and bottom detector layers is represented by subscripts in Eq. 1, embodiments are not limited to multi-layer detectors. For example, kV switching detectors may be used according to some embodiments to obtain multiple images in rapid succession. In such case, Eq. 1 may equivalently be written with subscripts [symbol] and 2, for example.) The measurements {y₁, y₂} represent pre-contrast injection, {y₃, y₄} represent post-injection, and the water distribution in the pre- and post-contrast measurements are represented by [symbol] ₁^{H}^{₂}^{O} and [symbol] ₂^{H}^{₂}^{O}, respectively. According to various embodiments, material decomposition involves processing the four measurements {y₁, y₂, y₃, y₄} in Eq. 1 to obtain the material path lengths { [symbol] ₁^{H}^{₂}^{O}, [symbol] ₂^{H}^{₂}^{O}, [symbol] ^{Co}, [symbol] [symbol] }. By way of non-limiting examples, two alternative approaches are disclosed: a lookup table approach and a model-based approach.

The lookup table approach according to various embodiments utilizes a staged decomposition, where projection-domain decomposition for the pre- and post-contrast measurements are conducted sequentially using two lookup tables:

$$\begin{matrix}\{ {\hat{l}}_{1}^{{H}_{2} O} , {\hat{l}}^{Ca} \} = {ℒ}_{{H}_{2} 0 , Ca} ( log {y}_{1} , {log}_{2} ) & \{ {\hat{l}}^{{H}_{2} O} , {\hat{l}}^{I} \} = {ℒ}_{{H}_{2} 0 , I} ( log {y}_{3} , {log}_{4} ; {l}^{Ca} )\end{matrix} \qquad ( 2 )$$

According to various embodiments, for the lookup table approach, pre-contrast measurements are first decomposed into water and calcium basis images using a lookup table that relates different density line integrals of water and calcium { [symbol] ^{H}^{₂}^{O}, [symbol] ^{Ca}} to the logarithm of the intensity measurements from the top and bottom detector layers {y̅₁,y̅₂}. The post-contrast decomposition may then be performed using a three-material lookup table that maps different density line integrals of water and iodine { [symbol] ^{H}^{₂}^{O}, [symbol] [symbol] } indexed at a fixed amount of calcium { [symbol] ^{Ca}} from the top and bottom detector measurements {y̅₃,y̅₄}.

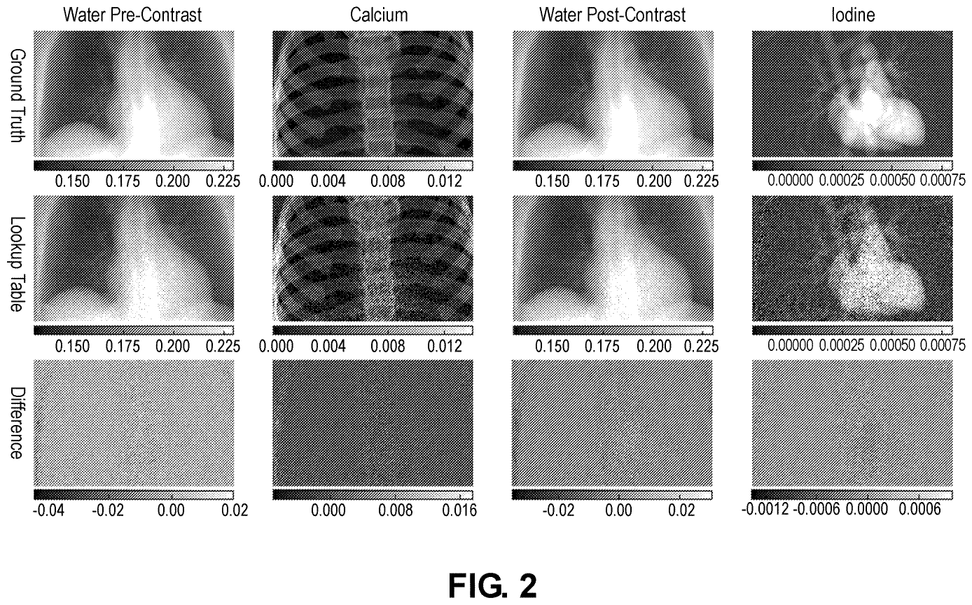

Note that the presence of the term [symbol] ^{Ca} in the second part of Eq. 2 may be solved using the first part of Eq. 2. Accordingly, embodiments that utilize lookup tables as disclosed herein may utilize a joint estimate to decompose the radiological measurements into material basis images. For example, the use of lookup tables can jointly perform three-material decomposition using four radiological measurements. A lookup table-based joint estimate that utilizes multiple radiological measurements simultaneously for three-material decomposition as disclosed herein is superior to prior art techniques that rely on separate individual material decompositions, e.g., as shown and described herein in reference to FIG. 2.

Decomposition using lookup tables can introduce noise, which can be corrected in post processing. Alternately, a model-based reconstruction algorithm for dual energy CT material decomposition that models the (potentially mismatched) geometry and scintillator blurs in the top and bottom layers may be used.

Various embodiments may use a model-based projection domain decomposition algorithm where the density line integrals, rather than the image-domain density maps, are estimated directly from the measurements. Consistent with this model-based approach, the four material images, [symbol] ={ [symbol] ₁^{H}^{₂}^{O}, [symbol] ^{Ca}, [symbol] ₂^{H}^{₂}^{O}, [symbol] [symbol] } are may be simultaneously using a penalized likelihood objective function, assuming that measurements, y={y₁, y₂, y₃, y₄} in each detector layer follow a multivariate Gaussian distribution with covariance K.

$$\hat{l} = arg {min}_{l} Φ ( l , y ) , where Φ ( l , y ) = \frac{1}{2} {( y - \overline{y} )}^{T} {K}_{y}^{- 1} ( y - \overline{y} ) + R ( l ) \qquad ( 3 )$$

According to various embodiments, the roughness penalty, R( [symbol] ) may be chosen to be a quadratic penalty, by way of non-limiting example, acting over the four nearest neighbors within each material image. According to various embodiments, a cross basis penalty may or may not be applied, e.g., instead of a roughness penalty. The penalty strength for each material image may be empirically set to 10⁵, 10⁶, 10⁵, and 10⁸. Various embodiments may use results from the lookup table method as initialization and 1000 iterations of a separable paraboloidal surrogates algorithm in the model-based approach. However, other initializations may be used, according to various embodiments.

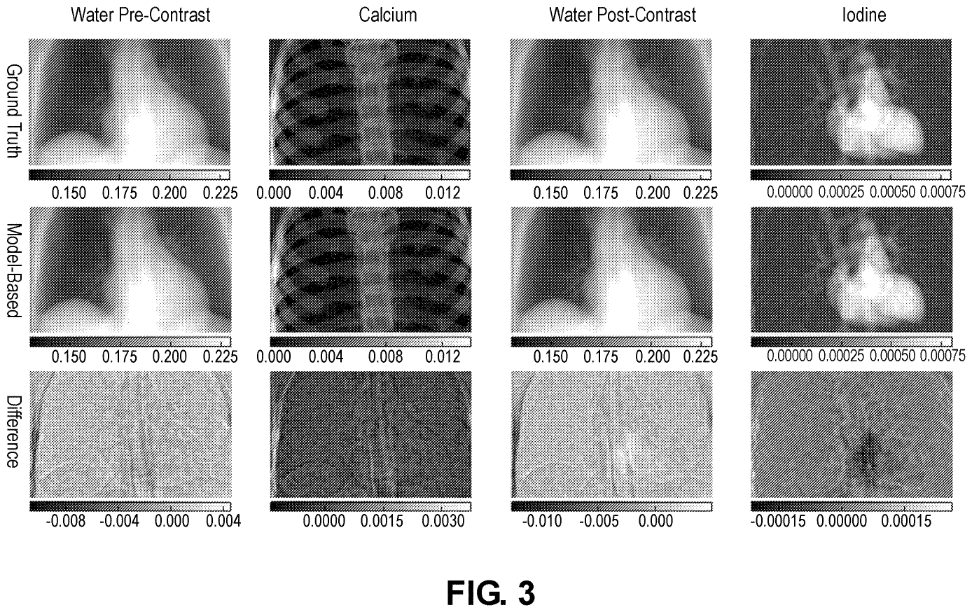

Accordingly, embodiments that utilize a model-based approach as disclosed herein may utilize a joint estimate to decompose the radiological measurements into material basis images. For example, the simultaneous estimate of the model-based approach can jointly perform three-material decomposition using four radiological measurements. A model-based joint estimate that utilizes multiple radiological measurements simultaneously for three-material decomposition as disclosed herein is superior to prior art techniques that rely on separate individual material decompositions, e.g., as shown and described herein in reference to FIG. 3.

A description of lookup table based and model-based reductions to practice follows, with comparisons to prior art approaches presented in reference to FIGS. 2 and 3. DSA images obtained using methods and systems consistent with this disclosure were compared to DSA images obtained using temporal subtraction and hybrid subtraction. In the former, pre- and post-contrast intensity measurements were log-transformed and subtracted. In the latter, and consistent with this disclosure, a water-calcium decomposition for both the pre- and post-contrast measurements was performed using the lookup table method. The two calcium images were then subtracted to obtain the iodine image.

For purposes of the reductions to practice, the XCAT phantom simulation package was used to generate the ground truth phantoms. The phantoms were generated in 3D and included ground truth density distributions of water, calcium, and iodine. For initial investigation, a perfect breath-hold was simulated where the diaphragm movement and AP expansion parameters were set to 0. A male thorax phantom was simulated at 20 time points through the default cardiac cycle. The 1^{st} point without iodine was used as the pre-contrast imaging, and the 15th time point with iodine enhanced vasculature was used as the post-contrast image.

Measurements were simulated using the forward model in Eq. 1. The source spectrum and spectral sensitivity of the detector was based on Spektr. Detector blurs for each layer were based on physical MTF measurements detailed in previous work. Independent Poisson noise was added to the measurements prior to the application of detector blur. For the initial investigation, magnification differences or misalignment in pixel grid between the top and bottom detectors were not simulated. Nevertheless, according to various embodiments, such differences can be addressed using an additional registration step.

To evaluate the various processing approaches, sample images are presented for qualitative comparison. For quantitative comparisons, an “oracle” scaling factor (based on the ground truth) was applied to the temporal and hybrid DSA images to obtain proper units of iodine. To assess accuracy of each method, the mean squared error and structural similarity index (SSIM) between the result and the ground truth are reported.

FIG. 2 depicts a four-material decomposition consistent with this disclosure using a lookup table approach, and FIG. 3 depicts a four-material decomposition consistent with this disclosure using a model-based approach. In particular, the four basis material density line integral maps are shown in FIG. 2 for the lookup table approach and in FIG. 3 for the model-based method. Material maps for water (pre- and post-injection), calcium, and iodine are shown in FIGS. 2 and 3, along with the ground truth and difference from truth images. Good separation among water, calcium, and iodine are observed in both cases. The lookup table method produces results that are noisy, consistent with material decomposition results from previous studies. The model-based method effectively mitigates noise and produce material images with low bias compared to the ground truth as seen from the difference images. The most challenging region for material separation is over the heart where the measurements are high noise and contain overlapping iodine, calcium, and water signals.

FIG. 4 depicts an improvement, according to reductions to practice, relative to prior art approaches. In particular, FIG. 4 depicts iodine images containing vessels in the left lung and compares the performance of traditional DSA methods (temporal and hybrid subtraction) with the results of the systems and methods disclosed herein. A zoomed region (rectangle) is shown where there is a significant change in the cardiac soft-tissue that yields subtraction artifacts in temporal DSA. Temporal subtraction has marked motion artifact due to cardiac motion. In comparison, major vessels around the heart are visible in the model-based material images, albeit at higher noise levels. The hybrid and lookup table methods are comparatively noisy. The SSIM and MSE were computed within the red ROI. From left to right, the SSIM to the ground truth is: 0.67, 0.77, 0.20, 0.19; the MSE to the ground truth is: 7.7e-9, 6.2e-10, 1.9e-8, 1.7e-8. Trends in these quantitative metrics are in agreement with visual inspection.

FIG. 5 is a flow diagram for a method 500 of generating a plurality of basis images for use in angiography, according to various embodiments. The method 500 may be performed using a system such as the system 100 shown and described herein in reference to FIG. 1.

At 502, the method 500 includes acquiring at least four radiological measurements of a target image region. The measurements may include: a pre-injection measurement set, including a first pre-injection measurement and a second pre-injection measurement, and a post-injection measurement set, including at least a first post-injection measurement and a second post-injection measurement. The measurements may be acquired using a projection x-ray system, such as is shown and described herein in reference to FIG. 1.

At 504, the method 500 includes decomposing, using a joint estimate, the radiological measurements into at least four basis images. The basis images may include: a pre-injection first material basis image, a post-injection first material basis image, a second material basis image, and a contrast agent basis image. The first material may be, e.g., calcium (showing bone) or water (showing soft tissue). The second material may be, e.g., calcium (showing bone) or water (showing soft tissue) and may be pre- or post-injection. For example, for imaging where the soft tissue is expected to move between pre-injection and post-injection measurements and the bone is expected to remain relatively motionless, the first material may be water and the second material may be calcium. For imaging where the bone is expected to move between pre-injection and post-injection measurements and the soft tissue is expected to remain relatively motionless, the first material may be calcium and the second material may be water. The actions of 504 may include performing three-material decomposition using all of the at least four radiological measurements jointly for the decomposition.

At 506, the method 500 includes providing at least the contrast agent basis image. The contrast agent basis image may be provided in any of a variety of ways. According to some embodiments, the contrast agent basis image may be provided by displaying it on a monitor. According to some embodiments, the contrast agent basis image may be provided electronically to a medical records storage system. According to some embodiments, the contrast agent basis image may be provided by sending it over a network such as the internet.

Thus, various embodiments embrace performing three material decomposition using a dual layer detector. The model-based method is shown to outperform the lookup table method in terms of noise and fidelity to the ground truth. Consistent with this disclosure, systems and methods can further refine both the lookup table and model-based algorithms for further image quality improvement (e.g., more sophisticated regularization).

Variations of embodiments of the invention will be apparent to those skilled in the art from consideration of the specification and practice of the embodiment disclosed herein.

For example, and without limitation, various embodiments can use other detector types, such as a triple layer detector or photon counting detectors. With such detectors, various embodiments can jointly estimate more material maps, which can allow systems and methods to account for calcium motion that are difficult to correct for (e.g., calcium plagues in the coronary artery).

Further still, and without limitation, methods and systems consistent with the current disclosure can use other images in the time series: Digital subtraction angiography usually acquires a series of images. Other than the pre-contrast image, other images prior to the current acquisition (including prior CT) can also be used to estimate basis material maps in the current acquisition.

It is intended that the specification and examples be considered as exemplary only, with a true scope and spirit of the invention being indicated by the following claims.

What is claimed is:

1. A method for generating a plurality of basis images for use in angiography, the method comprising:
acquiring at least four radiological measurements of a target image region, wherein the at least four radiological measurements include:
a pre-injection measurement set including at least a first pre-injection measurement and a second pre-injection measurement; and
a post-injection measurement set including at least a first post-injection measurement and a second post-injection measurement;
decomposing, using a joint estimate, the at least four radiological measurements into at least four basis images, the at least four basis images comprising: a pre-injection first material basis image, a post-injection first material basis image, a second material basis image, and a contrast agent basis image, wherein the decomposing the at least four radiological measurements into at least four basis images comprises performing at least three-material decomposition using all of the at least four radiological measurements; and
providing at least the contrast agent basis image.

2. The method of claim 1, wherein the first material comprises water, and wherein the second material comprises calcium.

3. The method of claim 1, wherein the contrast agent comprises iodine.

4. The method of claim 1,
wherein the acquiring comprises acquiring using a multi-energy detector,
wherein the multi-energy detector comprises a dual-layer flat panel detector, wherein the dual-layer flat panel detector includes at least a top layer detector and a bottom layer detector,
wherein the first pre-injection measurement comprises a pre-injection bottom layer detector measurement and wherein the second pre-injection measurement comprises a pre-injection top layer detector measurement, and
wherein the first post-injection measurement comprises a post-injection bottom layer detector measurement and wherein the second post-injection measurement comprises a post-injection top layer detector measurement.

5. The method of claim 4, wherein the performing at least three-material decomposition using all of the at least four radiological measurements comprises computing a plurality of line integrals of material density, wherein the plurality of line integrals of material density include: line integrals of first material density, line integrals of second material density, and line integrals of contrast agent density.

6. The method of claim 4, wherein the performing at least three-material decomposition using all of the at least four radiological measurements comprises:
decomposing the pre-injection measurement set into at least the pre-injection first material basis image and the second material basis image using a first lookup table; and
decomposing the post-injection measurement set into at least the post-injection first material basis image and the contrast agent basis image using a second lookup table,
wherein the first lookup table maps line integrals of first material density and line integrals of second material density to pre-injection intensity measurements from the top layer detector and the bottom detector layer, and
wherein the second lookup table comprises a three-material lookup table that maps the line integrals of first material density and line integrals of contrast agent density to post-injection intensity measurements from the top layer detector and the bottom detector layer, indexed according to the line integrals of second material density.

7. The method of claim 4, wherein the performing at least three-material decomposition using all of the at least four radiological measurements comprises:
estimating all of the at least four basis images simultaneously using a penalized likelihood objective function.

8. The method of claim 7, wherein the performing at least three-material decomposition using all of the at least four radiological measurements further assumes that the at least four radiological measurements of the target image region follow a multivariate Gaussian distribution with a covariance.

9. The method of claim 1, wherein the acquiring comprises acquiring using a triple-layer flat panel detector.

10. The method of claim 1, wherein the acquiring comprises acquiring using a photon counting detector.

11. The method of claim 1,
wherein the at least four radiological measurements further includes an additional measurement obtained prior to the post-injection measurement set, and
wherein the decomposing the at least four radiological measurements into at least four basis images further comprises performing at least three-material decomposition using all of the at least four radiological measurements and the additional measurement.

12. The method of claim 1, further comprising providing at least one of the pre-injection first material basis image, the post-injection first material basis image, or second material basis image.

13. A system for generating a plurality of basis images for use in angiography, the system comprising:
at least one computer;
at least one electronic storage device;
the at least one electronic storage device storing instructions that, when executed by the at least one computer, cause the at least one computer to generate a plurality of basis images by performing actions comprising:
acquiring at least four radiological measurements of a target image region, wherein the at least four radiological measurements include:
a pre-injection measurement set including at least a first pre-injection measurement and a second pre-injection measurement; and
a post-injection measurement set including at least a first post-injection measurement and a second post-injection measurement;
decomposing, using a joint estimate, the at least four radiological measurements into at least four basis images, the at least four basis images comprising: a pre-injection first material basis image, a post-injection first material basis image, a second material basis image, and a contrast agent basis image, wherein the decomposing the at least four radiological measurements into at least four basis images comprises performing at least three-material decomposition using all of the at least four radiological measurements; and
providing at least the contrast agent basis image.

14. The system of claim 13, wherein the first material comprises water, and wherein the second material comprises calcium.

15. The system of claim 13, wherein the contrast agent comprises iodine.

16. The system of claim 13, further comprising a multi-energy detector,
wherein the acquiring comprises acquiring using the multi-energy detector,
wherein the multi-energy detector comprises a dual-layer flat panel detector,
wherein the dual-layer flat panel detector includes at least a top layer detector and a bottom layer detector,
wherein the first pre-injection measurement comprises a pre-injection bottom layer detector measurement and wherein the second pre-injection measurement comprises a pre-injection top layer detector measurement, and
wherein the first post-injection measurement comprises a post-injection bottom layer detector measurement and wherein the second post-injection measurement comprises a post-injection top layer detector measurement.

17. The system of claim 16, wherein the performing at least three-material decomposition using all of the at least four radiological measurements comprises computing a plurality of line integrals of material density, wherein the plurality of line integrals of material density include: line integrals of first material density, line integrals of second material density, and line integrals of contrast agent density.

18. The system of claim 16, wherein the performing at least three-material decomposition using all of the at least four radiological measurements comprises:
decomposing the pre-injection measurement set into at least the pre-injection first material basis image and the second material basis image using a first lookup table; and
decomposing the post-injection measurement set into at least the post-injection first material basis image and the contrast agent basis image using a second lookup table,
wherein the first lookup table maps line integrals of first material density and line integrals of second material density to pre-injection intensity measurements from the top layer detector and the bottom detector layer, and
wherein the second lookup table comprises a three-material lookup table that maps the line integrals of first material density and line integrals of contrast agent density to post-injection intensity measurements from the top layer detector and the bottom detector layer, indexed according to the line integrals of second material density.

19. The system of claim 16, wherein the performing at least three-material decomposition using all of the at least four radiological measurements comprises:
estimating all of the at least four basis images simultaneously using a penalized likelihood objective function.

20. The system of claim 19, wherein the performing at least three-material decomposition using all of the at least four radiological measurements further assumes that the at least four radiological measurements of the target image region follow a multivariate Gaussian distribution with a covariance.

21. The system of claim 13, further comprising a triple-layer flat panel detector, wherein the acquiring comprises acquiring using the triple-layer flat panel detector.

22. The system of claim 13, further comprising a photon counting detector, wherein the acquiring comprises acquiring using the photon counting detector.

23. The system of claim 13,
wherein the at least four radiological measurements further includes an additional measurement obtained prior to the post-injection measurement set, and
wherein the decomposing the at least four radiological measurements into at least four basis images further comprises performing at least three-material decomposition using all of the at least four radiological measurements and the additional measurement.

24. The system of claim 1, wherein the actions further comprise providing at least one of the pre-injection first material basis image, the post-injection first material basis image, or second material basis image.
